(19) United States
(12) Patent Application Publication
(10) Pub. No.: US 2025/0297072 A1
(43) Pub. Date: Sep. 25, 2025

(54) COLOR CHANGEABLE (METH)ACRYLATE COMPOSITIONS AND METHODS OF USING THE SAME

(71) Applicant: Henkel AG & Co. KGaA, Duesseldorf (DE)

(72) Inventors: ChongYang Sun, Shanghai (CN); Joseph Zuohe Wang, Shanghai (CN); HeQiang Zhang, Shanghai (CN)

(21) Appl. No.: 19/229,455

(22) Filed: Jun. 5, 2025

Related U.S. Application Data
(63) Continuation of application No. PCT/CN2022/139640, filed on Dec. 16, 2022.

(51) Int. Cl. C08J 5/18; C08K 5/09; C08K 5/3447; C08K 5/47; C08K 11/00; G01N 21/64; G01N 33/44

(52) U.S. Cl. CPC C08J 5/18; C08K 5/09; C08K 5/3447; C08K 5/47; C08K 11/00; G01N 21/64; G01N 33/442; C08J 2433/12; C08K 2201/002

(57) ABSTRACT

Curable compositions for use in or as adhesives, sealants and/or coatings are disclosed herein. The compositions include a (meth)acrylate monomer, a (meth)acrylate terminated resin, an effective amount of a photoinitiator together with a dye substantially dissolved in the system which imparts good dyeing property in the (meth)acrylate composition. Upon curing, a resultant cured composition has a stable second color and obvious color change. The invention also relates to a method of detecting curing degree of the polymerization of the compositions.

### TECHNICAL FIELD

The present disclosure relates to color changeable (meth)acrylate compositions, which change visible color upon exposure to luminous radiation, along with related methods of using the same. The color changeable (meth)acrylate compositions can be used, for example, to detect the curing degree of the color changeable (meth)acrylate compositions, or to stably enhance visibility of cured film made from the color changeable (meth)acrylate compositions and provide non-destructive methods for inspection, to identify the presence of uncured and/or cured films, and to ensure proper coverage of the coating on an article.

### BACKGROUND OF THE INVENTION

Curing is a chemical process employed in polymer chemistry and process engineering that produces the toughening or hardening of a polymer material by cross-linking of polymer chains. Cured polymers are utilized as sealants, adhesives and coatings, as well as in a multitude of other uses in various industries. After the components of a curable components have been brought together under reactive conditions, the curing reaction proceeds at a given speed until curing has been completed at all available reactive sites. In luminous radiation curing process, the curing speed is usually a function of the luminous energy intensity, the luminous energy density and film thickness. When complete crosslinking of polymer chains has occurred, the polymer is referred to as having been “cured.” Cure monitoring is, for example, an essential component for the control of the manufacturing process of composite polymer materials. The curing degree is also impacted by the geometry of the film and substrate, the kind of equipment used for generating the luminous radiation, such as ultraviolet radiation, etc. The ability to identify curing degree, curing uniformity, as well as the onset of curing and the progress of curing, is highly needed.

Acrylate polymers make excellent coating and sealant systems because they have desirable electrical and physical properties, are resistant to fungus growth, have a long life, low or no exotherm during cure, and have little or no shrinkage during cure. Due to more strict regulations control in volatile solvents emission, the need in coating industry for high solid or solvent less/solvent free system increase. Acrylate polymer compositions which are 100% solids are known and have a rapid cure at a relatively low conversion energy demand. Such acrylate polymer compositions are cured by luminous radiation, especially ultraviolet radiation or by electron beam exposure. These are all reasons why it is important to have the ability to know when a curable composition is cured so that extra energy usage could be avoided, and the cost could be kept to a minimum.

US patent application U.S. Pat. No. 5,302,627A (Field etc) published a method of adding a dye with a visible color to an ultraviolet radiation curable silicone-containing polymeric composition which contains a photoinitiator which generates free radicals upon exposure to ultraviolet radiation produces a composition which changes visible color upon exposure to ultraviolet radiation. This visible color change indicates that the composition is cured. The cure indication is useful for compositions curable by ultraviolet radiation in the electronics and electrical industry. However, good indication property needs plenty of dye, which shows undesired curing inhibition.

US patent U.S. Pat. No. 6,890,399 (Wojciak etc) disclosed compositions including a (meth)acrylate component together with a dye substantially dissolved in the (meth)acrylate component which imparts a first color to the (meth)acrylate component. Upon curing, a resultant cured composition has a second color. The invention also relates to a method of detecting substantially full cure of the polymerization of the compositions. However, anthraquinone and/or xanthene dyes exhibit an inherent photobleaching property, and do not therefore provide a reliable indication that curing degree.

### SUMMARY OF THE INVENTION

It is therefore the object of the present invention to overcome the above-mentioned drawbacks by providing color changeable (meth)acrylate compositions which have good chromaticity property before curing, good color stability and noticeable color-changed effect after curing, as well as good strength property.

It has been surprisingly found that cured products prepared from the color changeable (meth)acrylate compositions of present invention, provides very good strength and noticeable color-changed effect. Meanwhile, it provided a non-destructive method to detect the curing degree of the (meth)acrylate compositions, or to stably enhance visibility of cured film made from the color changeable (meth)acrylate compositions and provide a reliable non-destructive method for inspection, to identify the presence of uncured and/or cured films, and to ensure proper coverage of the thin film coating on an article.

According to one aspect, the present invention relates to a color changeable (meth)acrylate composition comprising: a (meth)acrylate monomer, a (meth)acrylate terminated resin, an effective amount of a photoinitiator, and a dye being selected from a group consisting of benzopyranone derivatives, wherein the benzopyranone derivatives being represented by the following formulas:

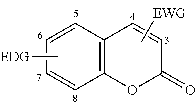

wherein, independently, EDG (electron-donating groups) being one or two substituents in any of the positions of 5, 6 and 7, which comprise one or more selected from the group consisting of substituted or unsubstituted amino group, substituted or unsubstituted alkylamino group, and substituted or unsubstituted alkenylamino group; EWG (electron-withdrawing groups) being one or two substituents in any of the positions of 3 and 4, which comprise one or more selected from the group of benzothiazole-2-yl, benzimidazol-2-yl, carbonitrile, and nitro.

According to one aspect, the present invention is directed to a cured composition comprising a cured product of the color changeable (meth)acrylate composition of present invention. The cured composition has a thickness of 0.12 to 1.0 mm. And the cured composition of present invention has a chromaticity of equal to or greater than 2 before curing, a chromaticity of equal to or greater than 2 after curing when subjected to ASTM E-308-2022 CIE lab test, and a color difference of equal to or greater than 4 after curing when subjected to ASTM D-2244-2015 CIE lab delta E test.

According to still another aspect, the present invention also relates a method of making a cured composition comprising the steps of a) providing the color changeable (meth)acrylate composition according to present invention; and b) photo curing the composition to form a cured composition having a second color; wherein prior to the step of curing, the color changeable (meth)acrylate composition has a fluorescent first color.

In still another aspect, the present invention is directed to a method of detecting curing degree of a curable composition comprising the steps of: a) providing a first article and an optional second article; b) providing, on a surface of the first article, the color changeable (meth)acrylate composition according to any one of claims 1-11, the color changeable (meth)acrylate composition has a fluorescent first color; c) optionally contacting a surface of the optional second article to the surface of the first article having the color changeable (meth)acrylate composition thereon; d) exposing the first article and the optional second article to cure conditions; and detecting the absence of fluorescence of the composition.

### DETAILED DESCRIPTION OF THE INVENTION

In the following passages the present invention is described in more detail. Each aspect so described may be combined with any other aspect or aspects unless clearly indicated to the contrary. In particularly, any feature indicated as being preferred or advantageous may be combined with any other feature or features indicated as being preferred or advantageous.

In the context of the present invention, the terms used are to be construed in accordance with the following definitions, unless a context dictates otherwise.

As used herein, the singular forms “a”, “an” and “the” include both singular and plural referents unless the context clearly dictates otherwise. For example, reference to “a filler” encompasses embodiments having one, two or more fillers. As used in this specification and the appended claims, the term “or” is generally employed in its sense including “and/or” unless the content clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or process steps.

The recitation of numerical end points includes all numbers and fractions subsumed within the respective ranges, as well as the recited end points.

Unless otherwise defined, all terms used in the disclosing the invention, including technical and scientific terms, have the meaning as commonly understood by one of the ordinary skills in the art to which this invention belongs to. By means of further guidance, term definitions are included to better appreciate the teaching of the present invention.

In the context of this disclosure, several terms shall be utilized.

The terms “polymer” is used herein consistent with its common usage in chemistry. Polymers are composed of many repeated subunits. The term “polymer” is used to describe the resultant material formed from a polymerization reaction.

As used herein, “M_{w}” refers to the weight average molecular weight and means the theoretical value as determined by Gel Permeation Chromatography (GPC) relative to linear polystyrene standards of 1.1 M to 580 Da and may be performed using Waters 2695 separation module with a Waters 2414 differential refractometer (RI detector).

As used herein, the term “cure” refers to exposing to radiation in any form, heating, or allowing to undergo a physical or chemical reaction that results in hardening or an increase in viscosity.

The term ‘light’ refers to electromagnetic radiation of any wavelength. The spectrum of electromagnetic radiation can be organized by decreasing wavelength and thus increasing energy into radio waves, microwaves, IR radiation, visible light, UV radiation, X-rays and gamma rays. Radiation within the UV spectrum can be further divided by wavelength into UVA (315-400 nm), UVB (280-315 nm) and UVC (100-280 nm). The most common types of UV sources are mercury and Light Emitting Diode (LED) lamps. The irradiation wavelength for mercury lamps ranges from 185 to 650 nm; however, they are being substituted by the less hazardous LED lamps, which irradiate at a much narrower range of 390-400 nm, the least energetic UV radiation. However, the recent developments in LED technology, emitting at 365-370 nm, have allowed the design of novel, powerful and efficient light sources that lead to the free radical and cationic photopolymerization of monomers, up to the synthesis of interpenetrating polymer networks (IPNs).

The term “oligomer” as used herein refers to relatively low molecular weight polymeric compounds which include at least two monomer units linked to each other. Desirably the oligomer includes from 2 to 1000 monomer units linked to each other, and more desirably 2 to 300 monomer units linked to each other.

The term “(meth)acryl” as used herein indicates acryl, methacryl or any combination thereof. Similarly, the term “(meth)acryloxy” indicates acryloxy, methacryloxy or any combination thereof; the term “(meth)acrylic acid” indicates acrylic acid, methacrylic acid or any combination thereof; the term “(meth)acrylate” indicates acrylate, methacrylate or any combination thereof; and the term “(meth)acrylamide” indicates acrylamide, methacrylamide or any combination thereof. The number of the (meth)acryl groups in the (meth)acrylate usable in the present invention is not particularly limited and can be one or more.

The CIELAB color space, also referred to as L*a*b*, is a color space defined by the International Commission on Illumination (abbreviated CIE). (Referring to CIELAB as “Lab” without asterisks should be avoided to prevent confusion with Hunter Lab). It expresses color as three values: L* for perceptual lightness and a* and b* for the four unique colors of human vision: red, green, blue and yellow. CIELAB was intended as a perceptually uniform space, where a given numerical change corresponds to a similar perceived change in color. While the LAB space is not truly perceptually uniform, it nevertheless is useful in industry for detecting small differences in color.

As used herein, the term “color change” means formation of a different color, loss of original color, or intensification of the original color, which can be indicated by color differences ΔE*_{ab}, between the reference and test sample. The color differences ΔE*_{ab} in L*a*b* can be approximated by treating each color as a point in a three-dimensional space (with three components: L*, a*, b*) and taking the Euclidean distance between them. The color-scale values L*, a* and b* were collected by colorimetric spectrometer (UltraScan PRO Spectrophotometer manufactured by HunterLab CORP.). The term “chromaticity” as used herein was indicated by the color difference between test sample and blank glass, which were calculated by using ASTM E308-2022.

As discussed previously, embodiments of the present disclosure are directed to a color changeable (meth)acrylate composition comprising a (meth)acrylate monomer, a (meth)acrylate terminated resin, an effective amount of a photoinitiator, and a specific dye.

### (Meth)Acrylate Monomer

Free radical addition polymerizable acrylate monomers are well known to those of ordinary skill in the art, and for purposes of the present invention conform to the general formula:

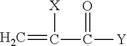

where X may be hydrogen, methyl, ethyl, or a halogen such as chlorine. Among these it is preferred that X be hydrogen or methyl. Y in the above formula may in turn be represented by the general formula:

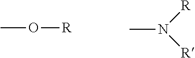

where R and R′ each represent hydrogen, alkyl groups of up to 8, and occasionally more, carbon atoms and such alkyl groups substituted with hydroxyl, amino, halo, or aryl substituents.

Those of ordinary skill in the art will readily recognize that the foregoing formulae define acrylic and methacrylic esters, acrylic and methacrylic amides, and variously substituted variations thereof as preferred acrylate monomers. Acrylic acid and methacrylic acid, and other carboxylic acid functional monomers comprise a special case hereinafter discussed in detail and are not intended for inclusion in the group of acrylate monomers here defined. The esters will include, as preferred members of the group, methyacrylate, methylmethacrylate, ethylacrylate, ethylmethacrylate, propylacrylate, propylmethacrylate, n-butylacrylate, n-butylmethacrylate, iso-butylacrylate, iso-butyl-methacrylate, and the like. In some circumstances, higher molecular weight esters, such as 2-ethylhexylacrylate and the like, and substituted, particularly hydroxy or amino-substituted alkyl acrylates and methacrylates, exemplified by, for example, 2-hydroxyethylacrylate and N-methylaminoethyl methacrylate, are useful and desirable. The amides include acrylamide and methacrylamide, and the corresponding N substituted amides.

Still other acrylate monomers may be employed, including acrylonitrile, methacrylonitrile, and diacrylic or dimethacrylic esters and amides, or ester-amides, resulting from the reaction of the acids with a diol, diamine, or the like, such as ethylene glycol dimethacrylate, ethylenediamine dimethacrylamide, acid salts of quarternized ammonium alkyl acrylates and methacrylates, and the like.

Mixtures of two or more of such acrylate monomers are also contemplated in the practice of the present invention.

Commercially available acrylate monomers include those sold as IBOA by Sartomer, DMAA by Sinopharm Chemical.

In embodiments of the present invention, the composition comprising, based on the total weight of the composition, at least 20-90 parts by weight, or at least 40-80 parts by weight, or at least 30-70 parts by weight, of the acrylate monomer. In some preferred embodiments, up to about 90 parts by weight, or up to about 80 parts by weight, or up to about 70 parts by weight, or up to about 50 parts by weight of the acrylate monomer. A preferred amount includes 30-70 prats by weight.

### (Meth)acrylate-Terminated Resin

The (meth)acrylate-terminated resins according to the present invention is not particularly limited. As used herein, the term “(meth)acrylate-terminated resin” refers to a resin having (meth)acrylate groups (i.e., unsaturated sites of carbon-carbon double bonds to an ester functional group —COOR) present at one or more, or all, of the terminal ends of the compound. As indicated, the acrylate-terminated resin has greater than two unsaturated sites.

(Meth)acrylate-terminated resins contain acrylate or methacrylate groups at their ends that will react during the free radical polymerization. In general, methacrylate-terminated resins are more efficient than acrylate-terminated resins for UV curing. Urethane acrylates, which contain a polyurethane backbone, are the most common, but epoxy, polyester or polyether backbones are also used.

As previously mentioned, the acrylate-terminated compound that is used in certain embodiments of the present invention is an acrylate-terminated resin having at least one or more than two unsaturated sites comprises a reaction product of reactants comprising: (i) an adduct of a polyisocyanate, wherein the adduct of a polyisocyanate comprises a reaction product of reactants comprising a polyisocyanate comprising greater than two isocyanate groups and a compound having groups reactive with the isocyanate groups of the polyisocyanate; and (ii) an active hydrogen-containing acrylate.

The acrylate-terminated resins also comprise an active hydrogen-containing acrylate. In certain embodiments, the active hydrogen-containing acrylate comprises hydroxyl-functional acrylates, amine-functional acrylates, or combinations thereof.

Example of commercially available acrylate-terminated resins are, for example, BR-3641AJ and BR-7432 GB from Bomar; and CN 959 from Sartomer.

In some embodiments of the present invention, the amount of the acrylate-terminated resins in the acrylate adhesive composition, based on the total weight of the composition, is from 5 to 80 parts by weight, and preferably from 30 to 70 parts by weight.

### Photoinitiator

The photoinitiator is generally used to initiate the unsaturated monomers or oligomer to conduct photo-polymerization. According to the present invention, the photoinitiator is used to initiate crosslinking polymerization of the (meth)acrylate monomers and acrylate-terminated resins.

When used, the photoinitiator may be chosen one or more from benzyl ketals, hydroxyl ketones, amine ketones and acylphosphine oxides, such as 2-hydroxy-2-methyl-1-phenyl-1-acetone, diphenyl (2,4,6-triphenylbenzoyl)-phosphine oxide, 2-benzyl-dimethylamino-1-(4-morpholinophenyl)-butan-1-one, benzoin dimethyl ketal dimethoxy acetophenone, a-hydroxy benzyl phenyl ketone, 1-hydroxy-1-methyl ethyl phenyl ketone, oligo-2-hydroxy-2-methyl-1-(4-(1-methylvinyl)phenyl)acetone, benzophenone, methyl o-benzyl benzoate, methyl benzoylformate, 2-diethoxy acetophenone, 2,2-d isec-butoxyacetophenone, p-phenyl benzophenone, 2-isopropyl thioxanthenone, 2-methylanthrone, 2-ethylanthrone, 2-chloroanthrone, 1,2-benzanthrone, benzoyl ether, benzoin ether, benzoin methyl ether, benzoin isopropyl ether, α-phenyl benzoin, thioxanthenone, diethyl thioxanthenone, 1,5-acetonaphthone, 1-hydroxycyclohexylphenyl ketone, ethyl p-dimethylaminobenzoate, and combinations thereof.

Mixtures of two or more of such photo-initiators are also contemplated in the practice of the present invention. Desirably, the photoinitiator is the combination of 2-hydroxy-2-methyl-1-phenyl-1-acetone and diphenyl (2,4,6-triphenylbenzoyl)-phosphine oxide.

If employed, the photoinitiator should be present in an amount sufficient to provide the desired rate of photopolymerization. This amount will be dependent in part on the light source, the thickness of the layer of the composition to be exposed to radiant energy and the extinction coefficient of the photoinitiator.

Photoinitiators may be used in an effective amount of about 0.1 percent by weight to about 5.0 percent by weight of the total composition, and desirably in about 1.0 percent by weight to about 4.0 percent by weight of the total composition.

Commercially available photoinitiator include those sold as Omnirad 184 and Omnirad TPO by IGM Resins.

### Dye

The dye according to the present comprise one or more being selected from a group consisting of benzopyranone derivatives, the benzopyranone derivatives being represented by the following formulas:

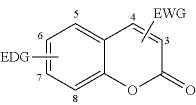

wherein, independently, EDG being electron-donating groups; EWG being electron-withdrawing groups.

In some embodiments, EDG could be one or more groups being each independently selected from a substituted or unsubstituted amino group, a substituted or unsubstituted alkylamino group, and a substituted or unsubstituted alkenylamino group. [6,7,8-ij]quinolizine group is not a suitable EDG for present disclosure.

In some preferred embodiments, EDG (electron-donating groups) being one or two substituents in any of the positions of 5, 6 and 7, more preferred in position 7, the EDG comprises tertiary amine groups.

In some preferred embodiments, the EDG (electron-donating groups) is substituent in the position 7 which being represented by the following formulas:

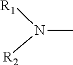

independently, R₁ being an alkyl group, an aryl group; R₂ being an alkyl group, an alkoxy group, an aryl group.

EWG (electron-withdrawing groups) being substituents in one or more positions of 3 and 4, more preferred in position 3, comprise one or more selected from the group of benzothiazole-2-yl, benzimidazol-2-yl, carbonitrile, nitro.

In some embodiments, the EWG (electron-withdrawing groups) is substituent in the position 3 which comprises benzothiazole-2-yl and benzimidazol-2-yl. In some preferred embodiments, the EWG (electron-withdrawing groups) further comprises substituent in positions 4 which comprises carbonitrile, nitro, and carboxyl.

In some embodiments, the dye is represented by the following formula:

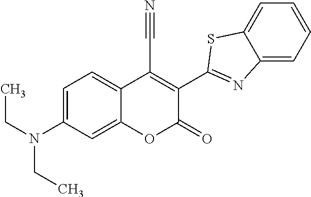 [SMILES: CCN(CC)c1ccc2c(C#N)c(-c3nc4ccccc4s3)c(=O)oc2c1]

Commercially available dye includes those sold as Macrolex Fluorescence Red G (3-(benzothiazol-2-yl)-7-(diethylamino)-2-oxo-2H-1-benzopyran-4-carbonitrile) by Lanxess.

In some other embodiments, the dye is represented by the following formula:

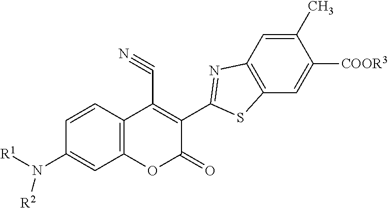

wherein R³ is C1-C11 alkyl, are also noteworthy, R¹ and R² independently are C1-C11 alkyl.

In some embodiments, the dye is represented by the following formulas:

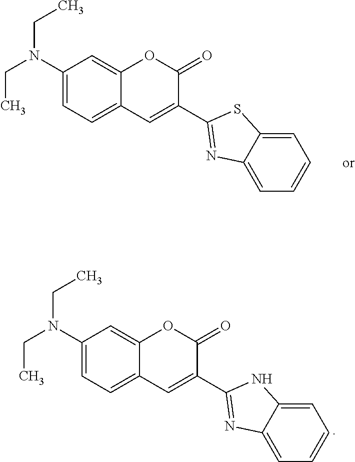 [SMILES: CCN(CC)c1ccc2cc(-c3nc4ccccc4s3)c(=O)oc2c1 ; CCN(CC)c1ccc2cc(-c3nc4ccccc4[nH]3)c(=O)oc2c1]

Alkyl substituted benzimidazole, such as 1-methyl-benzimidazole-2-yl is not suitable for present disclosure.

In some embodiments, the total amount of dye, based on the total weight of the (meth)acrylate composition (except dye), is at least 13 ppm, or at least 14 ppm, or at least 15 ppm, or at least 17 ppm, or at least 20 ppm, or at least 25 ppm, or at least 30 ppm, or at least 40 ppm, or at least 50 ppm. The total amount of dye is at most 200 ppm, or at most 180 ppm, or at most 150 ppm, or at most 100 ppm, or at most 90 ppm. The dye may be used in amounts of about 13 ppm to about 250 ppm of the total composition, and desirably in about 13 ppm to about 150 ppm, or 15 ppm to 100 ppm, or 20 ppm to 100 ppm, of the total composition.

Commercially available dye includes those sold as Coumarin 6 (3-(2-benzothiazolyl)-7-(diethylamino)-2H-1-benzopyran-2-one) and Coumarin 7 (3-(1H-Benzimidazol-2-yl)-7-(diethylamino)-2-benzopyrone) by J&K Chemical.

For more accurate curing indicating, the overlap between the initiator and light source must preferably not coincide with the absorption peaks of other components in the photopolymerization, such as monomers or dyes. Otherwise, in systems where there is overlap, higher light intensities and photoinitiator concentrations are often used, which may impact the strength of the system. It was found surprisingly, the (meth)acrylate compositions and use methods thereof in present invention provide a stable and accurate method to detect the curing degree and provide a non-destructive method for inspection, to identify the presence of uncured and/or cured films, and to ensure proper coverage of the coating on an article.

### Optional Additives

If desired, the composition of the invention can contain one or more optional adjuvants such as pigments, inhibitors, accelerators, photo-stabilizer, thermal stabilizers, viscosity modifiers, levelling agents, surfactants thickeners, plasticizers, and other ingredients. Those skilled in the art would realize the detailed examples of each type of the additives. Preferably, the total amount of optional additives, based on the total weight of the composition, is from 0 to 5 wt. %, more preferably 0 to 2 wt. %, particularly preferred 0 to 1 wt. %. In one particular embodiment the composition does not comprise any additives.

### Preparing Method of the Composition

Preparing methods according to the present invention also include obtaining one component luminous curable acrylate compositions according to the present invention and identify the coverage of uncured and/or cured composition on an article or detect the curing degree of the color changeable (meth)acrylate compositions.

In some embodiments of the present invention, the color changeable (meth)acrylate compositions are preferably prepared by steps of:
a) Mixing (Meth)acrylate monomer, photoinitiator and (meth)acrylate terminated resin together using a speed mixer
b) adding dye then the solution was again mixed to get homogeneous solution.

In some embodiments of the present invention, the color changeable (meth)acrylate compositions are used as a thin film. The (meth)acrylate composition of present invention could be used at a film thickness of equal to or greater than 0.12 mm, or 0.13 mm, or 0.15 mm. The thinner the film thickness is, the more difficult it is to achieve required color changed effect and chromaticity property. The (meth)acrylate composition of present invention could be used at a film thickness of equal to or less than 1.0 mm, or 0.8 mm, or 0.6 mm, or 0.5 mm, or 0.4 mm, or 0.0.3 mm. Some preferred film thickness range comprises 0.12 to 0.5 mm, or 0.12 to 0.4 mm, or 0.15 to 0.3 mm.

In some embodiments, UV energy also has impact on the chromaticity and color changed effect. For the (meth)acrylate composition films of present disclosure, the UV energy is higher than 1.5 J/cm². In some embodiments, to obtain a good chromaticity property after curing, the UV energy is less than 16 J/cm².

### LISTING OF EMBODIMENTS

1. A color changeable (meth)acrylate composition comprising
   a (meth)acrylate monomer,
   a (meth)acrylate terminated resin,
   an effective amount of a photoinitiator, and
   a dye being selected from a group consisting of benzopyranone derivatives,
   wherein the benzopyranone derivatives being represented by the following formulas:

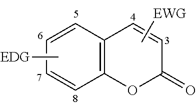

wherein, independently,
   EDG (electron-donating groups) being one or two substituents in any of the positions of 5, 6 and 7, which comprise one or more selected from the group consisting of substituted or unsubstituted amino group, substituted or unsubstituted alkylamino group, and substituted or unsubstituted alkenylamino group;
   EWG (electron-withdrawing groups) being one or two substituents in any of the positions of 3 and 4, which comprise one or more selected from the group of benzothiazole-2-yl, benzimidazol-2-yl, carbonitrile, and nitro.
2. The composition of embodiment 1, wherein the EDG (electron-donating groups) comprises tertiary amine groups.
3. The composition of any one of preceding embodiments, wherein the EDG (electron-donating groups) is substituent in the position 7 which being represented by the following formulas:

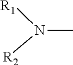

independently,
R₁ being an alkyl group, an aryl group;
R₂ being an alkyl group, an alkoxy group, an aryl group.
4. The composition of any one of preceding embodiments, wherein the EWG (electron-withdrawing groups) is substituent in the position 3 which comprises benzothiazole-2-yl and benzimidazol-2-yl.
5. The composition of any one of preceding embodiments, wherein the EWG (electron-withdrawing groups) further comprises carbonitrile, nitro, and carboxyl substituent in positions 4.
6. The composition of any one of preceding embodiments, wherein the (meth)acrylate monomer comprises polyethylene glycol di(meth)acrylates, bisphenol-A di(meth)acrylates, tetrahydrofurane (meth)acrylates and di(meth)acrylates, citronellyl acrylate and citronellyl methacrylate, hydroxypropyl (meth)acrylate, hexanediol di(meth)acrylate, trimethylol propane tri(meth)acrylate, tetrahydrodicyclopentadienyl (meth)acrylate, ethoxylated trimethylol propane triacrylate, triethylene glycol acrylate, triethylene glycol methacrylate, and combinations thereof.
7. The composition of any one of preceding embodiments, wherein the (meth)acrylate terminated resin comprises (meth)acrylate terminated epoxy, (meth)acrylate terminated urethane, (meth)acrylate terminated polyether, (meth)acrylate terminated polyester, (meth)acrylate terminated acrylate.
8. The composition of any one of preceding embodiments, wherein the dye is present in an amount of 13 ppm to 150 ppm based on the total amount of the composition.
9. The composition of any one of preceding embodiments, wherein the dye is present in an amount of 15 to 100 ppm based on the total amount of the composition.
10. The composition of any one of preceding embodiments, wherein said dye comprises 3-(benzothiazol-2-yl)-7-(diethylamino)-2-oxo-2H-1-benzopyran-4-carbonitrile, 3-(2-benzothiazolyl)-7-(diethylamino)-2H-1-benzopyran-2-one, 3-(1H-Benzimidazol-2-yl)-7-(diethylamino)-2-benzopyrone.
11. The composition of anyone of preceding embodiments, further comprising a member selected from the group consisting of pigments, stabilizers, accelerators, fillers, opacifiers, thickeners, viscosity modifiers, adhesion promoters, inhibitors, thixotrophy conferring agents, tougheners, anti-oxidizing agents, anti-reducing agents, and combinations thereof.
12. A cured composition comprising a cured product of the color changeable (meth)acrylate composition according to any one of preceding embodiments.
13. The cured composition of embodiment 12 has a thickness of 0.12 to 1.0 mm.
14. The cured composition of any one of embodiments 12 to 13 has a thickness of 0.15 to 0.3 mm.
15. The cured composition of any one of embodiments 12 to 14 has a chromaticity of equal to or greater than 2 before curing, a chromaticity of equal to or greater than 2 after curing when subjected to ASTM E-308-2022 CIE lab test, and a color difference of equal to or greater than 4 after curing when subjected to ASTM D-2244-2015 CIE lab delta E test.
16. A method of making a cured composition comprising the steps of:
   a) providing the color changeable (meth)acrylate composition according to any one of embodiments 1-11; and
   b) photo curing the composition to form a cured composition having a second color;
   wherein prior to the step of curing, the color changeable (meth)acrylate composition has a fluorescent first color.
17. The method of embodiment 16, wherein after the step of curing, the cured color changeable (meth)acrylate composition is substantially free of the first color and fluorescence.
18. The method of any one of embodiments 16 to 17, wherein the step of photo curing comprises ultraviolet photocuring.
19. The method of any one of embodiments 16 to 18, wherein the step of ultraviolet curing with the light source energy of equal to or greater than 1.5 J/cm².
20. The method of any one of embodiments 16 to 19, wherein the cured composition has a film thickness of 0.12 to 1.0 mm.
21. A method of detecting curing degree of a curable composition comprising the steps of
   a) providing a first article and an optional second article;
   b) providing, on a surface of the first article, the color changeable (meth)acrylate composition according to any one of embodiments 1-11, the color changeable (meth)acrylate composition has a fluorescent first color;
   c) optionally contacting a surface of the optional second article to the surface of the first article having the color changeable (meth)acrylate composition thereon
   d) exposing the first article and the optional second article to cure conditions; and detecting the absence of fluorescence of the composition.

### EXAMPLES

The present invention will be further described and illustrated in detail with reference to the following examples. The examples are intended to assist one skilled in the art to better understand and practice the present invention, however, are not intended to restrict the scope of the present invention. All numbers in the examples are based on weight unless otherwise stated.

### Raw Materials

| Description/function | Product name | Source |
|---|---|---|
| monomer | IBOA | Sartomer |
| monomer | DMAA | Sinopharm Chemical |
| photoinitiator | Omnirad 184 | IGM Resins |
| photoinitiator | Omnirad TPO | IGM Resins |
| acrylics resin | BR-3641AJ | Bomar |
| acrylics resin | BR-7432GB | Bomar |
| acrylics resin | CN 959 | Sartomer |
| adhesion promoter | Silquest A-187 | Momentive Performance |
| | | Materials |
| dye | Neutral red | Sinopharm Chemical |
| dye | Cresol red | Sinopharm Chemical |
| dye | Coumarin 30 | J&K Chemical |
| dye | Coumarin 343 | J&K Chemical |
| dye | Coumarin 6 | J&K Chemical |
| dye | Coumarin 7 | J&K Chemical |
| dye | Marcolex Fluorescent | Lanxess |
| | Red G | |
| dye | Methyl violet | Sinopharm Chemical |
| dye | Acid Red 18 (CAS No. | Sinopharm Chemical |
| | 2611-82-7) | |
| dye | Sudan Orange 220 (CAS | Sinopharm Chemical |
| | No. 842-07-9) | |
| dye | Solvent Green 3 (CAS | Sinopharm Chemical |
| | No. 128-80-3) | |
| dye | Solvent Blue 36 (CAS | Sinopharm Chemical |
| | No. 14233-37-5) | |
| dye | Acid Blue 1 (CAS No. | Sinopharm Chemical |
| | 116-95-0) | |
| dye | Solvent Red 24 (CAS | Sinopharm Chemical |
| | No. 85-83-6) | |

* All raw materials are directly used without any special treatment.

### Example 1

### <Preparation of Color Changeable (Meth)Acrylate Composition>

For the preparation of acrylate composition E1, approximately 60 grams of an aliphatic urethane acrylate oligomer (BR-3641AJ), 36 grams of acrylate monomer, 2 grams of photoinitiator (Omnirad TPO) and 2 grams of photoinitiator (Irgacure184) were mixed together by a speedmixer (sold under the trade designation “DAC 400 FV SPEEDMIXER by Hauschild Engineering, Germany). Subsequently, 3 mg of dye (Coumarin 6) was added into the system and mixed by Speedmixer until to get a homogeneous solution.

### <Curing of Color Changeable (Meth)Acrylate Composition>

### <Preparation of Test Specimens for Color-Scale Values Testing>

a) Wiping all glass specimens (with dimension of 1 mm*50 mm*80 mm) by Isopropyl alcohol and drying the specimens under room temperature (23±2° C.).
b) Applying about 2 grams of the adhesive composition onto one glass with two pieces of gap spacer with a specified diameter. The detailed diameter is descried in below example tables.
c) Assembling the second glass, pressing the glass together to ensure the adhesive spreads out evenly. Testing the color-scale values L*, a* and b* before curing. Blank Glass is used as standard (values L*, a* and b* are 96.6094, −0.2498 and 0.1278, separately under the same condition).
d) Setting the UV light using an ultraviolet light instrument for the specified intensity. The detailed light energy and intensity is listed in below example tables.
e) Curing the glass assembly under the light in the exposure area for the required exposure time specified in below example tables. Testing the color-scale values L*, a* and b* after curing.

### <Preparation of Test Specimens for Bonding Strength Testing>

a) Wiping all polycarbonate specimens with dimension of 6.0 mm*25.4 mm*25.4 mm by Isopropyl alcohol and dry it under room temperature (23±2° C.).
b) Applying about 0.03 grams of the adhesive compositions onto one glass with two pieces of gap spacers with a specified diameter. The detailed diameter is listed in below example tables.
c) Assembling the second polycarbonate specimen, the two polycarbonate specimens press form an angel of 180°, then pressing them together to ensure the adhesive spreads out evenly.
d) Setting the UV light using an ultraviolet light instrument (Loctite UVALOC 1000, Henkel China) for the specified intensity. The detailed light energy and intensity is listed in below example tables.
e) Curing the polycarbonate assembly under the light in the exposure area for the required exposure time specified in below example tables.

### Example 2-9, and CE1-CE13

The compositions of E2 to E9, CE1 to CE13 were prepared in reference to Example 1. More details are listed in below result part.

The uncured/cured color changeable (meth)acrylate composition samples were subjected to below tests.

### Test Methods

### <Bonding Strength>

The bonding strength of the (meth)acrylate adhesive composition of the present invention was assessed by ASTM D4501-01 (Mar. 10, 2001), shear strength of adhesive bonds between rigid substrates by the block-shear method.

### <Chromaticity and Calculation of Color Differences>

The CIE L*a*b* color coordinates of uncured and cured test specimens of the (meth)acrylate adhesive compositions of the present invention were assessed by ASTM E-308-2022, Standard Practice for Computing the Colors of Objects by Using the CIE System.

Chromaticity is indicated by the Color differences (ΔE*_{ab}) between cured/uncured test specimens and blank glass.

Color change is indicated by the Color differences (ΔE*_{ab}) between uncured test specimens and cured test specimens, which may be calculated by ASTM D 2244-2022, Calculation of Color Differences from Instrumentally Measured Color Coordinates.

Color differences (ΔE*_{ab}) provides a value indicating the overall difference between two colors (e.g., color difference between uncured and cured examples). It does not provide any color-related data such as which color is lighter/darker, redder/greener, more blue/more yellow. The color difference (ΔE*_{ab}) can be calculated by using the mathematical theorem of Pythagoras:

$$Δ {E}_{ab}^{*} ( color differences ) = \sqrt{{( {L}_{B}^{*} - {L}_{S}^{*} )}^{2} + {( {a}_{B}^{*} - {a}_{S}^{*} )}^{2} + {( {b}_{B}^{*} - {b}_{S}^{*} )}^{2}}$$

Where color coordinates L_{B}*, a_{B}*and b_{B}* refer to the test specimen, and L_{S}*, a_{S}* and b_{S}* refer to the reference or standard. A small ΔE*_{ab} value between uncured and cured examples implies that the colors are close before and after curing.

Regarding color change, ΔE*_{ab} (presenting roughly color differences or noted by visible color change between uncured and cured examples) results are recorded and ranked as follows:
Undetectable: lower than 4;
Detectable: equal to or greater than 4.

Regarding chromaticity, the minimum ΔE*_{ab} (color difference between cured/uncured test specimens and blank glass) for seeing a visible color is about at least 2, then ΔE*_{ab} can be used to show the visual identifiability or the coverage of acrylate compositions.

TABLE 1

| Product name | E1 | E2 | E3 | CE1 | CE2 | CE3 | CE4 | CE5 | CE6 | CE7 | CE8 | CE9 | CE10 | CE11 | CE12 |
|---|---|---|---|---|---|---|---|---|---|---|---|---|---|---|---|
| IBOA (g) | 18 | 18 | 18 | 18 | 36 | 36 | 18 | 36 | 18 | 18 | 18 | 36 | 20 | 18 | 18 |
| DMMA (g) | 18 | 18 | 18 | 18 | | | 18 | | 18 | 18 | 18 | | 20 | 18 | 18 |
| BR-7432GB (g) | | | | | | 60 | | | | | | 60 | | | |
| BR-3641AJ (g) | 60 | 60 | 60 | 60 | | | 60 | | 60 | 60 | 60 | | 60 | 60 | 60 |
| CN 959 (g) | | | | | 60 | | | 60 | | | | | | | |
| Omnirad TPO (g) | 2 | 2 | 2 | 2 | 2 | 2 | 2 | 2 | 2 | 2 | 2 | 2 | | 2 | 2 |
| Omnirad 184 (g) | 2 | 2 | 2 | 2 | 2 | 2 | 2 | 2 | 2 | 2 | 2 | 2 | | 2 | 2 |
| Coumarin 6 | 3 | | | | | | | | | | | | | | |
| (milligram) | | | | | | | | | | | | | | | |
| Coumarin 7 | | 3 | | | | | | | | | | | | | |
| (milligram) | | | | | | | | | | | | | | | |
| Macrolex | | | 3 | | | | | | | | | | 3 | | |
| Fluorescent | | | | | | | | | | | | | | | |
| red G | | | | | | | | | | | | | | | |
| (milligram) | | | | | | | | | | | | | | | |
| Coumarin 30 | | | | | | | | | | | | | | 3 | |
| (milligram) | | | | | | | | | | | | | | | |
| Coumarin 343 | | | | | | | | | | | | | | | 3 |
| (milligram) | | | | | | | | | | | | | | | |
| Acid blue 1 | | | | 3 | | | | | | | | | | | |
| (milligram) | | | | | | | | | | | | | | | |
| Acid red 18 | | | | | 3 | | | | | | | | | | |
| (milligram) | | | | | | | | | | | | | | | |
| Cresol red | | | | | | 3 | | | | | | | | | |
| (milligram) | | | | | | | | | | | | | | | |
| Solvent Blue 36 | | | | | | | 3 | | | | | | | | |
| (milligram) | | | | | | | | | | | | | | | |
| Neutral red | | | | | | | | 3 | | | | | | | |
| (milligram) | | | | | | | | | | | | | | | |
| Solvent Red 24 | | | | | | | | | 3 | | | | | | |
| (milligram) | | | | | | | | | | | | | | | |
| Solvent Green 3 | | | | | | | | | | 3 | | | | | |
| (milligram) | | | | | | | | | | | | | | | |
| Sudan Orange 220 | | | | | | | | | | | 3 | | | | |
| (milligram) | | | | | | | | | | | | | | | |
| Methyl violet | | | | | | | | | | | | 3 | | | |
| (milligram) | | | | | | | | | | | | | | | |

Table 1 shows formulations of the (meth)acrylate composition E1-E3 and CE1-CE12.

TABLE 2

| Items | E1 | E2 | E3 | CE1 | CE2 | CE3 | CE4 | CE5 | CE6 | CE7 | CE8 | CE9 | CE10 | CE11 | CE12 |
|---|---|---|---|---|---|---|---|---|---|---|---|---|---|---|---|
| | Film thickness: 1.0 mm; UV Energy: 7.6 J/cm² @ UVA; Light Source: LOCTITE Flood | | | | | | | | | | | | | | |
| | system with 365 nm LED lamp | | | | | | | | | | | | | | |
| Δ E_{ab}* | 53.6 | 38.3 | 37.9 | 13.1 | 3.4 | 18.6 | 8.7 | 19.4 | 18.5 | 11.2 | 17.5 | 1.5 | 10.7 | 15.6 | 29.2 |
| (uncured | | | | | | | | | | | | | | | |
| samples vs. | | | | | | | | | | | | | | | |
| blank glass) | | | | | | | | | | | | | | | |
| Δ E_{ab}* | 15.4 | 14.0 | 10.4 | 4.4 | 3.6 | 12.8 | 10.0 | 10.9 | 9.3 | 10.3 | 11.7 | 1.5 | 11.4 | 14.0 | 27.2 |
| (cured | | | | | | | | | | | | | | | |
| samples vs. | | | | | | | | | | | | | | | |
| blank glass) | | | | | | | | | | | | | | | |
| Δ E_{ab}* | 38.6 | 24.3 | 41.3 | 10.9 | 4.5 | 7.2 | 14.4 | 8.6 | 15.7 | 15.0 | 8.7 | 0.7 | 0.7 | 2.1 | 2.1 |
| (cured | | | | | | | | | | | | | | | |
| samples vs. | | | | | | | | | | | | | | | |
| uncured | | | | | | | | | | | | | | | |
| samples) | | | | | | | | | | | | | | | |
| | Film thickness: 0.2 mm; UV Energy: 5.7 J/cm² @ UVA; Light Source: LOCTITE Flood | | | | | | | | | | | | | | |
| | system with 365 nm LED Lamp | | | | | | | | | | | | | | |
| Δ E_{ab}* | 13.1 | 9.6 | 8.8 | 2.4 | 1.3 | 5.8 | 2.2 | 3.4 | 3.9 | 2.8 | 3.7 | — | — | — | — |
| (uncured | | | | | | | | | | | | | | | |
| samples vs. | | | | | | | | | | | | | | | |
| blank glass) | | | | | | | | | | | | | | | |
| Δ E_{ab}* | 2.4 | 3.2 | 2.7 | 1.3 | 0.8 | 0.8 | 2.1 | 1.7 | 1.4 | 2.2 | 1.3 | — | — | — | — |
| (cured | | | | | | | | | | | | | | | |
| samples vs. | | | | | | | | | | | | | | | |
| blank glass) | | | | | | | | | | | | | | | |
| Δ E_{ab}* | 11.3 | 6.5 | 9.7 | 1.4 | 0.5 | 5.0 | 2.5 | 1.9 | 3.6 | 3.0 | 2.6 | — | — | — | — |
| (cured | | | | | | | | | | | | | | | |
| samples vs. | | | | | | | | | | | | | | | |
| uncured | | | | | | | | | | | | | | | |
| samples) | | | | | | | | | | | | | | | |

Table 2 shows testing results of the (meth)acrylate composition E1-E3 and CE1-CE12.

In Examples 1 to 3, the (meth)acrylate compositions were prepared according to the formulations of color changeable (meth)acrylate compositions provided by the present invention. These formulations especially included the specific benzopyranone derivative dye, (meth)acrylate monomer, (meth)acrylate terminated resin and photoinitiator. It can be seen that when the contents of the claimed components of the present invention are within certain ranges, dye can act synergistically with acrylate system, such that the prepared (meth)acrylate compositions have good and stable chromaticity and noticeable color changed effect. When the produced film thickness is less than 1.0 mm, especially less than 0.3 mm, the prepared (meth)acrylate composition films still have good and stable chromaticity and noticeable color changed effect.

In Comparative examples 1 to 9 and 11 to 12, the (meth)acrylate compositions were prepared by a comparative dye. The prepared (meth)acrylate composition films don't have desired noticeable color changed effect, especially when the film thickness is less than 1.0 mm. In Comparative example 10, the (meth)acrylate composition doesn't comprise photoinitiator, the prepared (meth)acrylate composition film can't be cured well. It can be seen that example 10 doesn't have significantly color changes after exposure to luminous radiation, which also proves the dye disclosed in present invention don't have photo bleach properties.

TABLE 3

| | E4 | E5 | E6 | E7 | E8 | E9 | CE13 |
|---|---|---|---|---|---|---|---|
| | Film thickness: 0.15 mm; UV Energy: 2 J/cm² @ UVA; Light | | | | | | |
| | Source: Loctite UVALOC 1000 with Mercury Lamp, Henkel | | | | | | |
| Product name | China | | | | | | |
| IBOA (g) | 23.0 | 23.0 | 23.0 | 23.0 | 23.0 | 23.0 | 23.0 |
| DMMA (g) | 14.3 | 14.3 | 14.3 | 14.3 | 14.3 | 14.3 | 14.3 |
| BR-7432GB (g) | 57.2 | 57.2 | 57.2 | 57.2 | 57.2 | 57.2 | 57.2 |
| Silquest A-187 | 2.0 | 2.0 | 2.0 | 2.0 | 2.0 | 2.0 | 2.0 |
| Omnirad TPO (g) | 1.0 | 1.0 | 1.0 | 1.0 | 1.0 | 1.0 | 1.0 |
| Omnirad 184 (g) | 2.5 | 2.5 | 2.5 | 2.5 | 2.5 | 2.5 | 2.5 |
| Macrolex Fluorescent | 2.0 | 3.0 | 4.0 | 5.0 | 10.0 | 15.0 | 1.2 |
| red G | | | | | | | |
| (milligram) | | | | | | | |
| Dye content/ppm | 20.0 | 30.0 | 40.0 | 50.0 | 100.0 | 150.0 | 12.0 |

Table 3 shows formulations of the (meth)acrylate composition E4-E9 and CE13.

TABLE 4

| Test item | E4 | E5 | E6 | E7 | E8 | E9 | CE13 |
|---|---|---|---|---|---|---|---|
| Δ E_{ab}* (uncured samples vs. blank | 5.8 | 8.3 | 9.7 | 10.8 | 21.8 | 32.4 | 3.3 |
| glass) | | | | | | | |
| film thickness 0.15 mm | | | | | | | |
| Δ E_{ab}* (cured samples vs. blank | 2.2 | 2.9 | 2.5 | 2.6 | 3.3 | 4.1 | 2.0 |
| glass) | | | | | | | |
| film thickness 0.15 mm | | | | | | | |
| Δ Ex_{ab}* (cured samples vs. uncured | 5.6 | 7.8 | 9.2 | 10.4 | 20.6 | 31.0 | 3.0 |
| samples) | | | | | | | |
| film thickness 0.15 mm | | | | | | | |
| Bonding Strength (MPa) | 35.7 | 37.6 | 35.2 | 36.0 | 34.1 | 36.4 | 34.7 |

Table 4 shows testing results of the (meth)acrylate compositions E4-E9 and CE13.

As the content of the dye went up, from 20 to 150 ppm, based on the total weight of the (meth)acrylate composition, E4 to E9 showed good and stable chromaticity and noticeable color changed effect at a thin film thickness of 0.15 mm. In Comparative example 13, the dye content is less than 10 ppm, thus, the prepared (meth)acrylate composition films don't have desired noticeable color changed effect or good chromaticity after curing, especially when the thin film thickness is 0.15 mm. Within required content range of dye, E4 to E9 showed that the bonding strength has no concern with the content of dye. Certainly, at the same film thickness, the higher the dye content is, the better the color change effect of cured (meth)acrylate film is.

TABLE 5

| | | UV Energy | | | |
|---|---|---|---|---|---|
| | | @ UVA | Δ E*_{ab} | Δ E*_{ab} | Δ E*_{ab} |
| | | (J/cm²) | (uncured | (cured | (cured |
| | | (Light | samples | samples | samples |
| | Film | source: | vs. | vs. | vs. |
| | thickness | LED | blank | blank | uncured |
| Example | (mm) | 365 nm) | glass) | glass) | samples) |
| E1 | 0.15 mm | 0 | 12.8 | | |
| | | 5.1 | | 2.8 | 10.5 |
| | | 15.2 | | 3.2 | 10.2 |
| E2 | 0.15 mm | 0 | 9.3 | | |
| | | 5.1 | | 4.0 | 5.5 |
| | | 15.2 | | 2.6 | 6.8 |
| E3 | 0.15 mm | 0 | 6.3 | | |
| | | 0.5 | | 6.6 | 0.4 |
| | | 1.0 | | 6.3 | 0.5 |
| | | 1.5 | | 2.4 | 4.4 |
| | | 5.1 | | 2.1 | 6.9 |
| | | 15.2 | | 2.9 | 7.5 |
| | 0.254 mm | 0 | 11.1 | | |
| | | 0.3 | | 9.0 | 2.1 |
| | | 1.0 | | 8.6 | 2.5 |
| | | 1.5 | | 5.5 | 5.8 |
| | | 2.0 | | 2.1 | 10.6 |
| | | 3.0 | | 2.5 | 11.4 |
| | | 5.1 | | 3.7 | 11.8 |

Table 5 shows testing results of the (meth)acrylate compositions E1-E3 under different curing conditions and at different film thickness.

It can be seen that the effect of UV energy has impact on the color changed effect and chromaticity property. For a certain film thickness, the higher the UV energy is, the better the color change effect of cured (meth)acrylate film is. When the UV energy is equal to or greater than 1.5 J/cm², the prepared (meth)acrylate composition films have desired noticeable color changed effect or good chromaticity after curing, when the thin film thickness is less than 0.3 mm. The thicker film, the higher required UV energy. From the testing result of E3 at 0.254 film thickness, high UV energy has impact on the chromaticity property after curing.

TABLE 6

| | | | | Δ E*_{ab} |
|---|---|---|---|---|
| | | Δ E*_{ab} | Δ E*_{ab} | (cured |
| | Film | (uncured | (cured | samples vs. |
| | thickness | samples vs. | samples vs. | uncured |
| Example | (mm) | blank glass) | blank glass) | samples) |
| E3 | 0.1 | 3.3 | 1.1 | 3.6 |
| | 0.15 | 6.3 | 2.1 | 6.9 |
| | 0.2 | 8.8 | 2.7 | 9.7 |
| | 0.254 | 11.1 | 3.7 | 11.8 |
| | 1.0 | 37.9 | 10.4 | 41.3 |

Table 6 shows testing results of the (meth)acrylate composition E3 at different film thickness.

It can be seen that even at a film thickness greater than 0.12 mm, such as 0.15 mm, the (meth)acrylate composition film has qualified color changed effect and chromaticity property. The (meth)acrylate composition of present invention could be used at a film thickness of greater than 0.12 mm, or 0.15 mm. The thinner the film thickness is, the more difficult it is to achieve required color changed effect and chromaticity property. The (meth)acrylate composition of present invention could be used at a film thickness of equal to or less than 1.0 mm.

TABLE 7
the effect of photoinitiator or photo bleach

| | | | Δ E*_{ab} | Δ E*_{ab} | Δ E*_{ab} |
|---|---|---|---|---|---|
| | | | (uncured | (cured | (cured |
| | Adhesive' | UV Energy @ | samples | samples | samples |
| | Film | UVA (J/cm2), | vs. | vs. | vs. |
| | thickness | light source: | blank | blank | uncured |
| Examples | (mm) | LED 365 nm | glass) | glass) | samples) |
| CE10 | 0.254 | 5.1 | 10.7 | 11.4 | 0.7 |

Table 7 is the testing result of CE10 at a film thickness of 0.254 mm. In Comparative example 10, the (meth)acrylate composition doesn't comprise photoinitiator, and the (meth)acrylate composition doesn't have significantly color changes after exposure to luminous radiation. That is the color changeable (meth)acrylate composition of present invention don't have photobleaching property.

What is claimed is:

1. A color changeable (meth)acrylate composition comprising
a (meth)acrylate monomer,
a (meth)acrylate terminated resin,
an effective amount of a photoinitiator, and
a dye being selected from a group consisting of benzopyranone derivatives,
wherein the benzopyranone derivatives being represented by the following formulas: 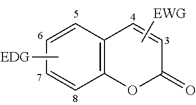
wherein, independently,
EDG (electron-donating groups) being one or two substituents in any of the positions of 5, 6 and 7, which comprise one or more selected from the group consisting of substituted or unsubstituted amino group, substituted or unsubstituted alkylamino group, and substituted or unsubstituted alkenylamino group;
EWG (electron-withdrawing groups) being one or two substituents in any of the positions of 3 and 4, which comprise one or more selected from the group of benzothiazole-2-yl, benzimidazol-2-yl, carbonitrile, and nitro.

2. The composition of claim 1, wherein the EDG (electron-donating groups) comprises tertiary amine groups.

3. The composition of claim 1, wherein the EDG (electron-donating groups) is substituent in the position 7 which being represented by the following formulas: 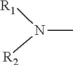
independently,
R₁ being an alkyl group, an aryl group;
R₂ being an alkyl group, an alkoxy group, an aryl group.

4. The composition of claim 1, wherein the EWG (electron-withdrawing groups) is substituent in the position 3 which comprises benzothiazole-2-yl and benzimidazol-2-yl.

5. The composition of claim 4, wherein the EWG (electron-withdrawing groups) further comprises carbonitrile, nitro, and carboxyl substituent in positions 4.

6. The composition of claim 1, wherein the (meth)acrylate monomer comprises polyethylene glycol di(meth)acrylates, bisphenol-A di(meth)acrylates, tetrahydrofurane (meth)acrylates and di(meth)acrylates, citronellyl acrylate and citronellyl methacrylate, hydroxypropyl (meth)acrylate, hexanediol di(meth)acrylate, trimethylol propane tri(meth)acrylate, tetrahydrodicyclopentadienyl (meth)acrylate, ethoxylated trimethylol propane triacrylate, triethylene glycol acrylate, triethylene glycol methacrylate, and combinations thereof.

7. The composition of claim 1, wherein the (meth)acrylate terminated resin comprises (meth)acrylate terminated epoxy, (meth)acrylate terminated urethane, (meth)acrylate terminated polyether, (meth)acrylate terminated polyester, (meth)acrylate terminated acrylate.

8. The composition of claim 1, wherein the dye is present in an amount of 13 ppm to 150 ppm based on the total amount of the composition.

9. The composition of claim 1, wherein the dye is present in an amount of 15 to 100 ppm based on the total amount of the composition.

10. The composition of claim 1, wherein said dye comprises 3-(benzothiazol-2-yl)-7-(diethylamino)-2-oxo-2H-1-benzopyran-4-carbonitrile, 3-(2-benzothiazolyl)-7-(diethylamino)-2H-1-benzopyran-2-one, 3-(1H-Benzimidazol-2-yl)-7-(diethylamino)-2-benzopyrone.

11. The composition of claim 1, further comprising a member selected from the group consisting of pigments, stabilizers, accelerators, fillers, opacifiers, thickeners, viscosity modifiers, adhesion promoters, inhibitors, thixotrophy conferring agents, tougheners, anti-oxidizing agents, anti-reducing agents, and combinations thereof.

12. A cured composition comprising a cured product of the color changeable (meth)acrylate composition according to claim 1.

13. The cured composition of claim 12 has a thickness of 0.12 to 1.0 mm.

14. The cured composition of claim 12 has a thickness of 0.15 to 0.3 mm.

15. The cured composition of claim 13 has a chromaticity of equal to or greater than 2 before curing, a chromaticity of equal to or greater than 2 after curing when subjected to ASTM E-308-2022 CIE lab test, and a color difference of equal to or greater than 4 after curing when subjected to ASTM D-2244-2015 CIE lab delta E test.

16. A method of making a cured composition comprising the steps of:
a) providing the color changeable (meth)acrylate composition according to claim 1; and
b) photo curing the composition to form a cured composition having a second color;
wherein prior to the step of curing, the color changeable (meth)acrylate composition has a fluorescent first color.

17. The method of claim 16, wherein after the step of curing, the cured color changeable (meth)acrylate composition is substantially free of the first color and fluorescence.

18. The method of claim 16, wherein the step of photo curing comprises ultraviolet photocuring.

19. The method of claim 18, wherein the step of ultraviolet curing with the light source energy of equal to or greater than 1.5 J/cm².

20. The method of claim 16, wherein the cured composition has a film thickness of 0.12 to 1.0 mm.

21. A method of detecting curing degree of a curable composition comprising the steps of
a) providing a first article and an optional second article;
b) providing, on a surface of the first article, the color changeable (meth)acrylate composition according to any claim 1, the color changeable (meth)acrylate composition has a fluorescent first color;
c) optionally contacting a surface of the optional second article to the surface of the first article having the color changeable (meth)acrylate composition thereon;
d) exposing the first article and the optional second article to cure conditions; and detecting the absence of fluorescence of the composition.
